(19) United States
(12) Patent Application Publication
(10) Pub. No.: US 2021/0338096 A1
(43) Pub. Date: Nov. 4, 2021

(54) MEASURING INSTRUMENT

(71) Applicant: Murata Manufacturing Co., Ltd., Nagaokakyo-shi (JP)

(72) Inventors: Jun Takagi, Nagaokakyo-shi (JP); Tomoki Takahashi, Nagaokakyo-shi (JP); Yoshie Kuramochi, Nagaokakyo-shi (JP); Hiroaki Togashi, Nagaokakyo-shi (JP); Kenji Tanaka, Nagaokakyo-shi (JP)

(21) Appl. No.: 17/376,719

(22) Filed: Jul. 15, 2021

Related U.S. Application Data
(63) Continuation of application No. PCT/JP2020/006148, filed on Feb. 18, 2020.

(30) Foreign Application Priority Data
Feb. 19, 2019 (JP) 2019-027512

(51) Int. Cl. A61B 5/0537

(52) U.S. Cl. CPC A61B 5/0537

(57) ABSTRACT

A measuring instrument is provided for inserting into an oral cavity with a narrow opening and that enables intraoral measurement in such a way as to relieve stress exerted on a probe part of the measuring instrument. The measuring device is configured to ensure that the probe part is accurately pressed against the oral mucosa of a person subjected to measurement. The measuring instrument includes a grip and a probe. The probe includes a measurement section and a joint forming a connection between the measurement section and grip. The measurement section is a tip region of the probe and provided with the sensor having an exposed measurement surface. A circuit board is disposed in the joint. The circuit board is an oscillation circuit board on which members forming an oscillation circuit are mounted. The oscillation circuit outputs an oscillatory signal corresponding to an electrical signal transmitted from the sensor.

### CROSS REFERENCE TO RELATED APPLICATIONS

The present application is a continuation of PCT/JP2020/006148 filed Feb. 18, 2020, which claims priority to Japanese Patent Application No. 2019-027512, filed Feb. 19, 2019, the entire contents of each of which are incorporated herein by reference.

### TECHNICAL FIELD

The present disclosure relates to a measuring instrument.

### BACKGROUND

In general, an intraoral moisture measuring instrument is described in International Publication No. 2004/028359 (hereinafter “Patent Document 1”) and is an example of instrument designed to be held by a person who conducts measurement on a subject, such as a medical patient. The intraoral moisture measuring instrument includes a probe and a capacitive sensor provided in a tip region of the probe and is configured to measure the moisture content of the subject of measurement in a state in which a measurement surface of the sensor is pressed against and parallel to the surface of the subject of measurement (e.g., tongue mucosa).

Using this design, it can be difficult for a person subjected to the measurement to stick out his or her tongue. In such a case, a person who conducts the measurement with the measuring instrument of Patent Document 1, for example, may experience difficulty in inserting the tip region of the probe into the oral cavity of the person subjected to measurement or in pressing the measurement surface of the sensor against and parallel to the tongue in the oral cavity. These problems may be averted by reducing the profile of the probe. On the downside, such a low-profile probe can be deformed by the application of stress when being pressed against the subject of measurement. This can lead to dispersion in the results of measurement.

### SUMMARY OF THE INVENTION

Accordingly, it is an object of the exemplary embodiments of the present disclosure to provide a high-precision, high-reliability measuring instrument that resolves the difficulty of inserting a measuring instrument into an oral cavity with a narrow opening and enables intraoral measurement in such a way as to relieve stress exerted on a probe part of the measuring instrument. The measuring instrument is also designed to ensure that the probe part is accurately pressed against the oral mucosa of a person subjected to measurement.

Thus, according to an exemplary embodiment, a measuring instrument includes a grip, a probe, and a circuit board. The probe includes a measurement section and a joint that forms a connection between the measurement section and the grip. The measurement section is a tip region of the probe and provided with a sensor having a measurement surface exposed at a first surface of the measurement section. The circuit board is disposed in the grip or the joint and has an oscillation circuit mounted thereon. The oscillation circuit is configured to output an oscillatory signal corresponding to an electrical signal transmitted from the sensor.

This configuration provides a high-precision, high-reliability measuring instrument that resolves the difficulty of inserting a measuring instrument into an oral cavity with a narrow opening. The measure instrument enables intraoral measurement in such a way as to relieve stress exerted on a probe part of the measuring instrument and ensures that the probe is accurately pressed against the oral mucosa of a person subjected to measurement.

The measuring instrument according to an exemplary aspect of the present disclosure offers an advantage in that the stress on the probe part will be relieved.

### BRIEF DESCRIPTION OF DRAWINGS

FIG. 1 is a schematic side view of a measuring instrument according to an exemplary embodiment.

FIG. 2 is a schematic plan view of the measuring instrument according to the exemplary embodiment.

FIG. 3 is an enlarged plan view of a sensor part placed in such a manner that a sensor surface of the sensor part is in the viewer's line of sight.

FIG. 4 is a schematic side view of a measuring instrument according to a modification of the exemplary embodiment.

FIG. 5 is a schematic side view of a measuring instrument according to another modification of the exemplary embodiment.

FIG. 6 is a schematic side view of a measuring instrument according to still another modification of the exemplary embodiment.

FIG. 7 is a schematic side view of a measuring instrument according to still another modification of the exemplary embodiment.

FIG. 8 is a schematic side view of a measuring instrument according to still another modification of the exemplary embodiment.

FIG. 9 is a schematic side view of a measuring instrument according to still another modification of the exemplary embodiment.

FIG. 10 is a schematic side view of a measuring instrument according to still another modification of the exemplary embodiment.

FIG. 11 is a schematic side view of a measuring instrument according to still another modification of the exemplary embodiment.

### DETAILED DESCRIPTION OF EMBODIMENTS

Hereinafter, an exemplary embodiment of the present disclosure will be described.

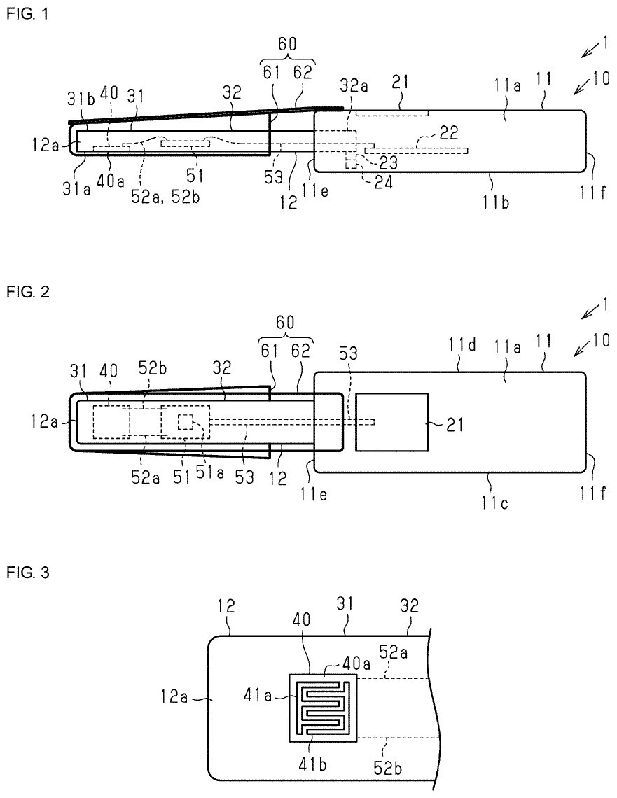

As illustrated in FIGS. 1 and 2, a measuring instrument 1 includes a main body 10 and a cover 60, which is fitted over the main body 10. The measuring instrument 1 can, for example, be an intraoral moisture measuring instrument that is constructed to measure the moisture content in the oral cavity.

The main body 10 includes a grip part 11 (also referred to as a “grip”) and a probe part 12 (also referred to as a “probe”). The grip part 11 is an end portion in the longitudinal direction of the main body 10. The probe part 12 is arranged at the other end portion in the longitudinal direction of the main body 10.

According to an exemplary aspect, the grip part 11 is substantially a rectangular parallelepiped and has a top surface 11a, a bottom surface 11b, a side surface 11c, a side surface 11d, an end surface 11e, and an end surface 11f. A display part 21 is in the top surface 11a of the grip part 11 to display, for example, results of measurement.

The probe part 12 protrudes through the end surface 11e, which is one of the end surfaces of the grip part 11. The probe part 12 in the present embodiment is a rectangular parallelepiped and is plate-like in shape, for example. The probe part 12 includes a measurement section 31 and a joint section 32 (also referred to as a “joint”). The measurement section 31 is a tip region of the probe part 12. The joint section 32 forms a connection between the measurement section 31 and the grip part 11.

The measurement section 31 is a rectangular parallelepiped and is plate-like in shape, for example. The measurement section 31 has a measurement surface 31a (e.g., a first surface) and a back surface 31b, which is opposite the measurement surface 31a.

As illustrated in FIG. 3, the measurement section 31 is provided with a sensor 40. The sensor 40 is flat and plate-like in shape. The sensor 40 has a measurement surface 40a, which is flat. The sensor 40 may, for example, be a capacitive sensor. As can be seen from the side view in FIG. 1, the measurement surface 40a of the sensor 40 is flush with the measurement surface 31a of the measurement section 31. As can be seen from FIG. 3, the measurement surface 40a of the sensor 40 is exposed at the measurement surface 31a of the measurement section 31.

As further shown, a pair of electrodes is disposed on the measurement surface 40a of the sensor 40. One electrode of the pair of electrodes is denoted by 41a, and the other electrode is denoted by 41b. The electrodes 41a and 41b can, for example, be comb teeth-shaped. The electrodes 41a and 41b act as capacitor electrodes in the exemplary embodiment. With the subject of measurement facing the measurement surface 40a, the subject and liquid in the surface of the subject act as a dielectric for the electrodes 41a and 41b. Moreover, the capacitance of the electrodes 41a and 41b varies in relation to the subject of measurement and the moisture content of the surface of the subject.

According to the exemplary aspect, the joint section 32 is a rectangular parallelepiped and is plate-like in shape. The joint section 32 and the measurement section 31 in the present embodiment are equal in thickness and are also equal in width.

Moreover, the probe part 12 in the present embodiment is swingably (or otherwise movably) supported by a shaft (not illustrated) of the grip part 11. Referring to FIG. 1, the joint section 32 has an end portion 32a, which is inserted in the grip part 11 and is joined to the grip part 11 by the shaft (not illustrated).

A circuit board 51 is disposed in the joint section 32. The circuit board 51 is connected to the electrodes 41a and 41b of the sensor 40 (see FIG. 3), with a pair of traces being laid between the circuit board 51 and the electrodes 41a and 41b. One trace of the pair of traces is denoted by 52a, and the other trace is denoted by 52b. The traces 52a and 52b preferably extend in parallel and apart from each other. This layout is conducive to reducing the parasitic capacitance between the traces 52a and 52b. The traces 52a and 52b in the present embodiment extend along the respective edges that are opposite in the direction of the width of the probe part 12.

Moreover, the circuit board 51 is an oscillation circuit board on which members forming an oscillation circuit 51a are mounted. The oscillation circuit 51a can, for example, be a CR oscillation circuit and outputs an oscillatory signal corresponding to an electrical signal transmitted from the sensor 40. More specifically, the oscillation circuit 51a outputs an oscillatory signal of the frequency corresponding to the value of the capacitance between the electrodes 41a and 41b of the sensor 40. The oscillation circuit 51a is connected to a main circuit board 22, with a harness 53 being laid therebetween. The main circuit board 22 is disposed in the grip part 11. The harness 53 comprises a plurality of lines. The lines forming the harness 53 include: a power supply line through which operating voltage is supplied from the grip part 11 to the oscillation circuit 51a; and a signal line through which an oscillatory signal from the oscillation circuit 51a is transmitted to the main circuit board 22.

The main circuit board 22 is a control circuit board on which a CPU and other members forming a control circuit for controlling the measuring instrument 1 are mounted. The control circuit is configured to determine the moisture content of the subject of measurement on the basis of the number of pulses of the signal output from the oscillation circuit 51a. The control circuit causes the display part 21 to display the determined moisture content.

The grip part 11 is provided with a force exerting member 23, which exerts force on the joint section 32 of the probe part 12. The force exerting member 23 may, for example, be an elastic member such as a spring or rubber. With the probe part 12 being swingably (or otherwise movably) supported by the grip part 11, the force exerting member 23 exerts force on the joint section 32 of the probe part 12 in one of the directions in which the probe part 12 is constructed to swing. The force may, for example, be exerted in a direction such that a distal end 12a of the probe part 12 swings to the side on which the measurement surface 31a of the measurement section 31 is located, as illustrated in FIG. 1.

The grip part 11 is provided with a switch part 24, which is turned on or off in response to a swing of the probe part 12. The switch part 24 is turned on when the probe part 12 swings in such a way as to resist the force exerted by the force exerting member 23. At the turn-on of the switch part 24, the control circuit may, for example, start the processing for determining the moisture content.

According to an exemplary aspect, the probe part 12 in the present embodiment is made from resin, with the measurement section 31 and the joint section 32 being incorporated in one.

The cover 60 includes a cover member 61 and a support member 62. The cover member 61 is in the form of a flat bag. The support member 62 is securely fixed to the cover member 61. The cover member 61 and the support member 62 are made from a transparent or translucent resin. The cover 60 is securely fixed in such a manner that the measurement section 31, which is the tip region of the probe part 12, is covered with the cover member 61. The cover member 61 keeps the tip region of the probe part 12, or more specifically, the sensor 40 in the measurement section 31 from direct contact with the subject of measurement.

Operation and Configuration

The following describes the operations and configuration of the measuring instrument 1.

The measuring instrument 1 includes the grip part 11 and the probe part 12. The probe part 12 includes the measurement section 31 and the joint section 32. The measurement section 31 is the tip region of the probe part 12. The joint section 32 forms a connection between the measurement section 31 and the grip part 11. The measurement section 31 is provided with the sensor 40. The measurement surface 40a of the sensor 40 is exposed at the measurement surface 31a of the measurement section 31.

The probe part 12 is, by and large, flat and plate-like in shape. The measurement section 31, which is the tip region of the probe part 12, is provided with the sensor 40. The circuit board connected to the sensor 40 is disposed in the joint section 32 of the probe part 12. The measurement section 31, which is the tip region of the probe part 12, is thin and can thus be easily inserted into the oral cavity of a person subjected to measurement. This resolves the difficulty of inserting a measuring instrument into an oral cavity with a narrow opening. The measurement surface 40a of the sensor 40 comes entirely into close contact with the surface of the subject of measurement (e.g., a patient). The dispersion in the results of measurement will be minimized accordingly. The easiness of inserting the measurement section 31, which is the tip region of the probe part 12, also facilitates the measurement of the moisture content in the oral cavity of a person who has difficulty in sticking out his or her tongue or in opening his or her mouth.

With the grip part 11 of the measuring instrument 1 being held by a person who conducts measurement, the measurement surface 40a of the sensor 40 is brought into close contact with a surface of the subject of measurement (e.g., the surface of the tongue of a person subjected to measurement). More specifically, the measurement section 31, which is the tip region of the probe part 12, is pressed against the tongue with a predetermined amount of force by the person who conducts measurement so that the measurement surface 40a of the sensor 40 comes into close contact with the surface of the tongue. Consequently, the measurement surface 40a of the sensor 40 comes entirely into close contact with the surface of the subject of measurement. The dispersion in the results of measurement will be minimized accordingly.

When the measurement section 31 is pressed against the subject of measurement, the pressing force is likely to cause the probe part 12, which is made from resin and is plate-like in shape, to become warped. This problem can be averted by the present embodiment, in which the circuit board 51 is disposed in the joint section 32 of the probe part 12. The reason for this is that the circuit board 51 is more rigid than the resinous exterior of the probe part 12 and thus relieves the stress exerted on the probe part 12.

The circuit board 51 in the joint section 32 of the probe part 12 is preferably adjacent to the sensor 40 in the measurement section 31. The stress exerted on the probe part 12 is greater in a site closer to the distal end of the probe part 12. Thus, the circuit board 51 close to the distal end of the probe part 12 is conducive to relieving the stress.

The probe part 12 of the measuring instrument 1 in the present embodiment is swingably supported by the grip part 11, and the grip part 11 is provided with the force exerting member 23, which exerts force on the probe part 12. When the measurement section 31 comes into contact with the subject of measurement, an end portion being part of the probe part 12 and held in the grip part 11 is subjected to pressure. The force exerting member 23 relieves the pressure exerted on the end portion being part of the probe part 12 and held in the grip part 11.

As described above, the present embodiment produces the following effects.

First, the measuring instrument 1 includes the grip part 11 and the probe part 12. The probe part 12 includes the measurement section 31 and the joint section 32, which forms a connection between the measurement section 31 and the grip part 11. The measurement section 31 is the tip region of the probe part 12 and provided with the sensor 40, which has the measurement surface 40a exposed at the measurement surface 31a of the measurement section 31. The circuit board 51 is disposed in the joint section 32. The circuit board 51 is an oscillation circuit board on which members forming the oscillation circuit 51a are mounted. The oscillation circuit 51a outputs an oscillatory signal corresponding to an electrical signal transmitted from the sensor 40. When the measurement section 31 is pressed against the subject of measurement, the pressing force is likely to cause the probe part 12, which is made from resin and is plate-like in shape, to become warped. The circuit board 51 disposed in the joint section 32 is more rigid than the resinous exterior of the probe part 12. The configuration described provides the measuring instrument 1, that is, a high-precision, high-reliability measuring instrument that resolves the difficulty of inserting a measuring instrument into an oral cavity with a narrow opening. The measuring instrument enables intraoral measurement in such a way as to relieve stress exerted on the probe part 12 and also ensures that the probe part 12 is accurately pressed against the oral mucosa of a person subjected to measurement.

Second, the measurement section 31, which is the tip region of the probe part 12, is thin and can thus be easily inserted into an oral cavity. The measurement surface 40a of the sensor 40 comes entirely into close contact with the surface of the subject of measurement. The dispersion in the results of measurement will be minimized accordingly.

Third, the probe part 12 is swingably supported by the grip part 11, and the grip part 11 is provided with the force exerting member 23, which exerts force on the probe part 12 in a direction in which the probe part 12 swings. When the measurement section 31 comes into contact with the subject of measurement, the end portion being part of the probe part 12 and held in the grip part 11 is subjected to pressure. The force exerting member 23 relieves the pressure exerted on the end portion being part of the probe part 12 and held in the grip part 11.

Fourth, the sensor 40 is a capacitive sensor. The oscillation circuit 51a mounted on the circuit board 51 is connected to the sensor 40, with a pair of traces, namely, the traces 52a and 52b being laid therebetween. The traces 52a and 52b extend in parallel and apart from each other. This layout is conducive to reducing the parasitic capacitance between the traces 52a and 52b. The traces 52a and 52b are thus less likely to affect the electrostatic capacity between the electrodes 41a and 41b of the sensor 40, and stable measurement is ensured accordingly.

Fifth, the grip part 11 is provided with the switch part 24 (also referred to as a “switch”), which is turned on or off in response to a swing of the probe part 12. The switch part 24 is turned on when the probe part 12 swings in such a way as to resist the force exerted by the force exerting member 23. At the turn-on of the switch part 24, the processing for determining the moisture content may be started.

### Modifications of the Exemplary Embodiment

The embodiment above may be implemented as follows.

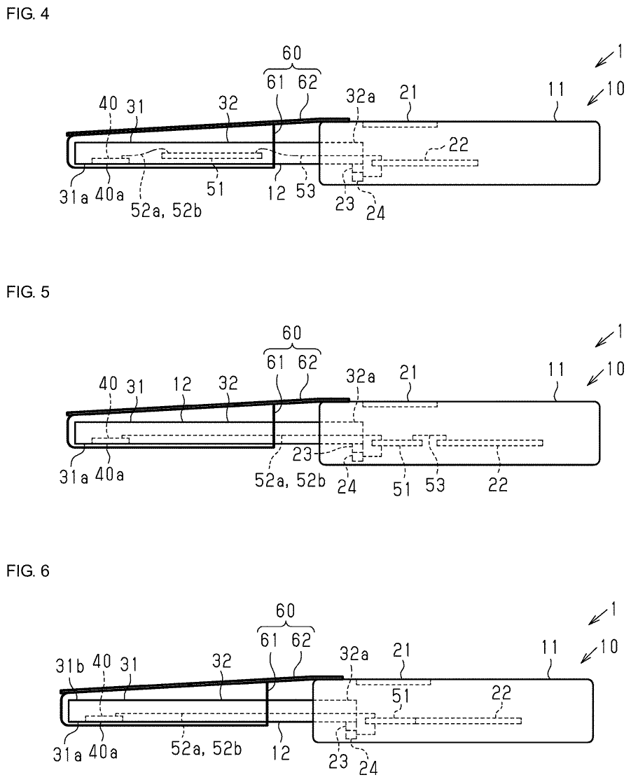

As illustrated in FIG. 4, the length of the circuit board 51 may be changed as appropriate. Increasing the length of the circuit board 51 is conducive to relieving the stress on the probe part 12 even further.

As illustrated in FIG. 5, the circuit board 51 may be disposed in the grip part 11. As illustrated in FIG. 6, the circuit board 51 and the main circuit board 22 may be disposed monolithically.

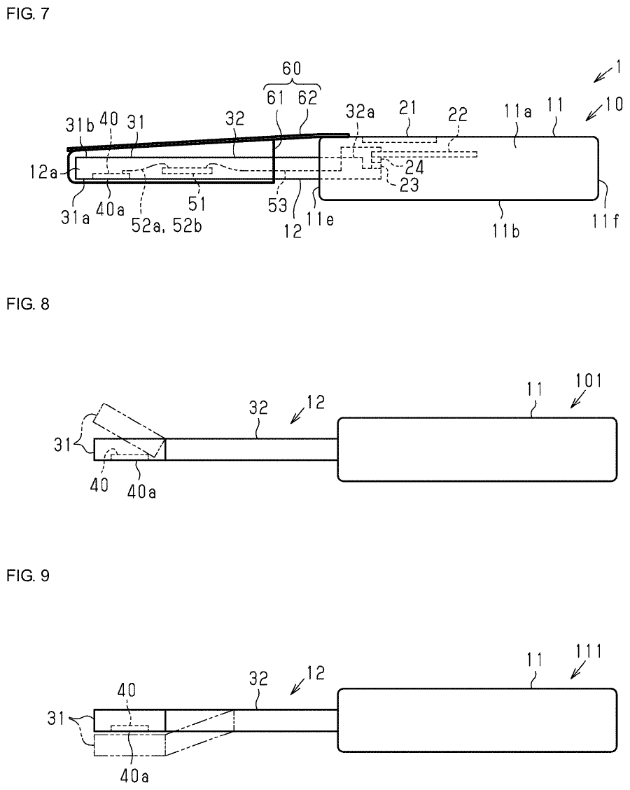

As illustrated in FIG. 7, the switch part 24 can be disposed on the main circuit board 22 in the grip part 11. The switch part 24 can be incorporated in the main circuit board 22. For example, the switch part 24 can be connected directly, electrically, and mechanically to the main circuit board 22. The force exerting member 23 can be incorporated in the probe part 12 (e.g., the joint section 32 of the probe part 12). For example, the force exerting member 23 can be supported directly and mechanically by the joint section 32 of the probe part 12.

The switch part 24 disposed on the main circuit board 22 offers an advantage in that the electrical configuration and/or the mechanical configuration of the measuring instrument 1 is simplified through elimination of wires forming an electrical connection between the switch part 24 and the main circuit board 22 and/or through the use of the main circuit board 22 as a support of the switch part 24. The simplified configuration advantageously improves the reliability of the measuring instrument 1.

Moreover, the switch part 24 may be turned on or off in the following manner. When the probe part 12 does not swing, or more specifically, when the measurement section 31 is not pressed against the subject of measurement, the force exerting member 23 incorporated in the probe part 12 (e.g., the joint section 32 of the probe part 12) exerts force in such a way as to turn off the switch part 24. The probe part 12 swings and moves away from the switch part 24 to cause the switch part 24 to be turned on. This configuration prevents an excessive load on the probe part 12 from being transmitted from the probe part 12 to the switch part 24 or to the main circuit board 22 and thus advantageously eliminates or reduces the possibility of breakage of the switch part 24 or the main circuit board 22.

As illustrated in FIGS. 8 to 11, the configuration of the probe part may be changed as appropriate. It is noted that the cover 60 is not illustrated in FIGS. 8 to 11.

FIG. 8 illustrates a measuring instrument 101, the probe part 12 of which is distinguishable by its measurement section 31 being tiltable relative to the joint section 32. The measurement section 31 of the probe part 12 of the measuring instrument 101 tilts such that the measurement surface 31a of the measurement section 31 and the measurement surface 40a of the sensor 40 come into close contact with the surface of the subject of measurement. The measurement surface 40a may, for example, be brought into close contact with the surface of the tongue in a manner so as to be less obstructed by front teeth on the lower jaw. It becomes also possible to bring the measurement surface 40a to close contact with, for example, the palatal mucosa adjacent to a second molar.

FIG. 9 illustrates a measuring instrument 111, the probe part 12 of which is distinguishable by its measurement section 31 being configured to undergo a translational motion relative to the joint section 32 in a direction perpendicular to the measurement surface 31a. The measurement section 31 of the probe part 12 of the measuring instrument 111 undergoes a translational motion such that the measurement surface 31a of the measurement section 31 and the measurement surface 40a of the sensor 40 come into close contact with the surface of the subject of measurement. As is the case with the measuring instrument 101 illustrated in FIG. 8, the measurement surface 40a may, for example, be brought into close contact with the surface of the tongue in a manner so as to be less obstructed by front teeth on the lower jaw. It becomes also possible to bring the measurement surface 40a into close contact with, for example, the palatal mucosa adjacent to a second molar.

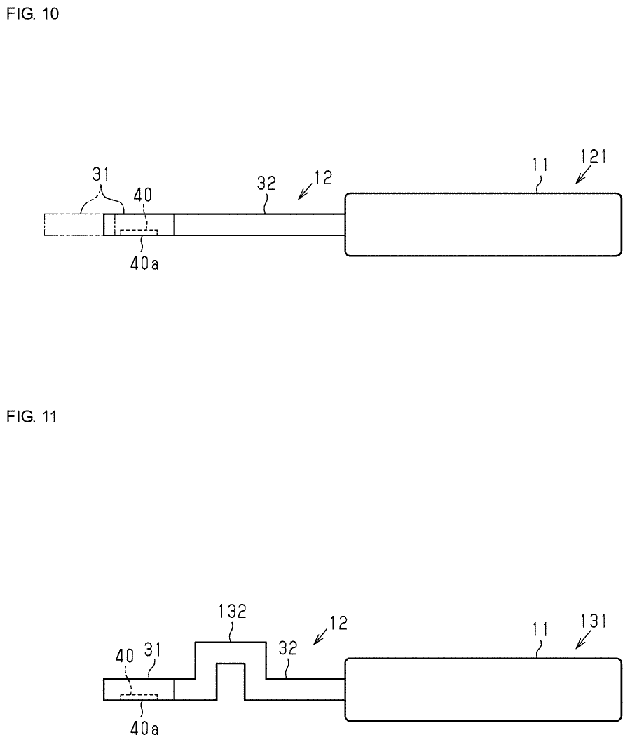

FIG. 10 illustrates a measuring instrument 121, the probe part 12 of which is distinguishable by its joint section 32 being configured for expanding and contracting in length. The joint section of the probe part 12 of the measuring instrument 121 expands in length such that the measurement section 31 comes into close contact with the surface of the subject of measurement in a state in which the grip part 11 is held away from the subject of measurement. This configuration is advantageous in conducting measurement on, for example, the tongue or posterior buccal mucosa of an infected patient.

FIG. 11 illustrates a measuring instrument 131, the probe part 12 of which is distinguishable by its joint section 32 including a bypass portion 132. The bypass portion 132 has a U-shape with an open bottom. The bypass portion 132 enables the measurement surface 31a of the measurement section 31 and the measurement surface 40a of the sensor 40 to come into close contact with the surface of the subject of measurement without being obstructed by, for example, front teeth on the lower jaw. In some embodiments, the joint section 32 including the bypass portion 132 is slightly curved.

Exemplary embodiments have been described above in which the measuring instrument is designed for intraoral measurement of the moisture content. In some embodiments, the measuring instrument is designed for extraoral measurement of the moisture content. It is noted that the cover 60 is optional. That is, the sensor 40 may be pressed directly against the subject of measurement to determine the moisture content.

Exemplary embodiments have been described above in which the measuring instrument is designed for measurement of the moisture content. In some embodiments, the measuring instrument is designed for measurement of the pH or oral bacteria or for measurement of the bloodstream or the level of oxygen in the blood. The measuring instrument may be designed for taking various kinds of measurements. Needless to say, the capacitive sensor in the embodiment above is to be replaced with a sensor appropriate to the subject of measurement concerned according to these alternative embodiments as would be appreciated to those skilled in the art.

### REFERENCE SIGNS LIST

1 measuring instrument

11 grip part

12 probe part

31 measurement section

31a measurement surface

32 joint section

40 sensor

40a measurement surface

51 circuit board

1. A measuring instrument having a longitudinal direction, the measuring instrument comprising:
a grip;
a probe including a measurement section and a joint section that forms a connection between the measurement section and the grip, the measurement section including a sensor having a measurement surface exposed in a direction perpendicular to the longitudinal direction; and
a circuit board disposed in the joint section and having an oscillation circuit mounted thereon, the oscillation circuit being configured to output an oscillatory signal corresponding to an electrical signal detected by the sensor, the circuit board having a principal surface that is parallel to the measurement surface.

2. The measuring instrument according to claim 1, wherein the probe is flat and plate-like in shape.

3. The measuring instrument according to claim 1, wherein the circuit board is more rigid than a resinous exterior of the probe.

4. The measuring instrument according to claim 1, wherein the circuit board disposed in the joint section of the probe is adjacent to a distal end of the probe.

5. The measuring instrument according to claim 1, wherein the joint section is configured to expand and contract in length.

6. The measuring instrument according to claim 1, wherein the joint section of the probe includes a bypass portion.

7. The measuring instrument according to claim 6, wherein the bypass comprises U-shape with an open bottom constructed to extend around teeth of a user when the probe is inserted into a mouth of the user.

8. The measuring instrument according to claim 1, wherein the probe is swingably supported by the grip, and the grip includes a force exerting member that is constructed to exert force on the probe in a direction in which the probe swings.

9. The measuring instrument according to claim 1, wherein the sensor is a capacitive sensor and the oscillation circuit is connected to the sensor with a pair of traces being laid therebetween.

10. The measuring instrument according to claim 9, wherein one trace of the pair of traces extends parallel to and away from the other trace.

11. The measuring instrument according to claim 9, wherein the pair of traces extend parallel and apart from each other.

12. The measuring instrument according to claim 1, wherein the grip includes a switch that is configured to turn on and off in response to a swing movement of the probe.

13. The measuring instrument according to claim 12, wherein the switch is disposed on a main circuit board in the grip.

14. The measuring instrument according to claim 13, wherein the probe is constructed to swing and move away from the switch to cause the switch to be turned on.

15. The measuring instrument according to claim 1, further comprising a pair of electrodes disposed on the measurement surface the sensor.

16. The measuring instrument according to claim 15, wherein the pair of electrodes comprise a comb teeth shape.

17. A measuring instrument extending in a lengthwise direction, the measuring instrument comprising:
a grip;
a probe that has a joint section that movably connects to the grip;
a measurement section disposed in the probe and including a sensor with a measurement surface that is exposed in a direction perpendicular to the lengthwise direction; and
a circuit board disposed in the joint section and having an oscillation circuit that is configured to output an oscillatory signal corresponding to an electrical signal detected by the sensor,
wherein the circuit board has a principal surface that extends in a direction parallel to the measurement surface.

18. The measuring instrument according to claim 17, wherein the probe is swingably supported by the grip, and the grip includes a force exerting member that is constructed to exert force on the probe in a direction in which the probe swings.

19. The measuring instrument according to claim 17, wherein the sensor is a capacitive sensor and the oscillation circuit is connected to the sensor with a pair of traces being laid therebetween.

20. The measuring instrument according to claim 19, wherein one trace of the pair of traces extends parallel to and away from the other trace.
